(19) United States
(12) Patent Application Publication
(10) Pub. No.: US 2002/0019013 A1
(43) Pub. Date: Feb. 14, 2002

(54) Combined resin method for high-speed synthesis of combinatorial libraries

(76) Inventors: Boliang Lou, Louisville, KY (US); Tawfik Gharbaoui, Poway, CA (US)

Correspondence Address: John E. Vanderburgh, 608 Sunset Road, Louisville, KY, 40206, US

(21) Appl. No.: 09/855,197

(22) Filed: May 14, 2001

Related U.S. Application Data
(63) Continuation-in-part of application No. 09/264,515, filed on Mar. 8, 1999.

(51) Int. Cl. G01N 33/53; C07D 241/04; C07D 243/12

(52) U.S. Cl. 435/7.1; 540/504; 544/385

(57) ABSTRACT

A method for high-speed parallel synthesis of combinatorial libraries is disclosed where two or more resins of dissimilar functionality are combined in the same reaction vessel in which a plurality of chemical reactions are carried out to create multiple compounds which are then sequentially and individually cleaved from the different resins under the appropriate cleavage conditions for each resin. As used herein resins are considered different when they exhibit different chemical activity in the presence of cleaving or releasing agents. The resins are different when the individual resins have either dissimilar polymeric backbones or dissimilar linkers or both and thus have a different chemical activity in the presence of a release or cleaving agent from the other resins in the reaction vessel.

This application is a continuation-in-part of co-pending application Ser. No. 09/264,515, entitled A COMBINED RESIN METHOD FOR HIGH-SPEED SYNTHESIS OF COMBINATORIAL LIBRARIES, filed Mar. 8, 1999 by Lou et al. and claims the benefit of its filing date under 35 U.S.C. 120.

### FIELD OF THE INVENTION

The present invention relates to a general method for high-speed parallel synthesis of combinatorial libraries based on the combined resin method. In contrast to the traditional parallel approach which usually create one compound in one vessel, the present method employs multiple resins in the same reaction vessel to create multiple compounds which are then sequentially cleaved from the resins under the appropriate conditions.

### BACKGROUND OF THE INVENTION

The use of solid phase synthesis techniques for the synthesis of polypeptides and oligonucleotides are well known in the art. More recently, the use of solid phase techniques for the synthesis of small organic molecules has become a major focus of research. Of prime importance has been the ability of solid phase techniques to be automated, with an attendant increase in compound throughput and efficiency in research. This has been exploited with great vigor in the area of pharmaceutical research where it has been estimated that 10,000 compounds must be synthesized and tested in order to find one new drug (Science, 259, 1564, 1993). The focus on combinatorial chemistry techniques to increase compound throughput has now become almost universal in the pharmaceutical and agricultural industries.

An additional aspect relates to the chemical diversity of the compound stocks that are available for screening in pharmaceutical companies in the search for new lead structures. These have tended to be limited to the classes of compounds previously investigated through medicinal chemical techniques within each company. Therefore the availability of new classes of molecules for screening has become a major need.

Combinatorial chemistry involves both the synthesis and screening of large sets of compounds, called libraries. The libraries themselves can be arrays of individual compounds or mixtures. Therefore, the synthetic approaches are also classified into two categories, including combinatorial synthesis of mixtures and parallel synthesis leading to individual compounds. For screening purposes it is also important that the formed compounds be synthesized in 1 to 1 mol ratios.

In the first approach to creating molecular diversity, the combinatorial synthesis comprises multiple reactions in one reaction vessel resulting in the generation of all possible product combinations from a set of reactants. The simplest manifestation of the approach is to allow several reagents to react in solution at the same time to form all possible products. Among the examples is the synthesis of a library of over 97,000 members by reaction of a mixture of amines with 9,9-dimethylxanthene-2,4,5,7-tetracarboxylic acid tetrachloride (Carell, T; et al. Angew. Chem. Int. Ed. Engl. 33, 2059). However, this approach is usually unproductive unless the reagents are few and their reactivities are well matched to approach formation of the various compounds in 1 to 1 mol ratios.

Another approach is the use of the portioning-mixing method or the split synthesis (Furka, A; et al. Int. J. Pept. Protein Res. 37, 487, 1991). As shown in FIG. 1, the synthesis is executed by repetition of three simple operations, including dividing the solid support into equal portions, reacting each portion individually with one of the building blocks and then homogeneously mixing the portions. As it can be seen, starting with a single substance the number of compounds is tripled after each coupling step. As illustrated in the synthesis of trimers, 27 different compounds are prepared in three pools. These compounds can be cleaved into solution and screened as soluble pools, or the ligands can remain attached to the beads and screened in immobilized form. However, biological screens performed on such large mixtures of soluble compounds can be ambiguous since the observed activity could be due to a single compound or to a combination of compounds acting either collectively or synergistically. The subsequent identification of specific biologically active members is challenging, since the numbers of compounds present in the pools and their often limited concentration deter their isolation and re-assay. Because of this, the identification of individual active compounds from the library requires the repetitive re-synthesis and retesting of the most active smaller subsets of the library until activity data are obtained on homogenous compounds. There is no direct method available to elucidate the chemical structures of large libraries of mixtures. However many methods have been developed to aid and accelerate the deconvolution process, including recursive deconvolution and multiple encoding approaches. There still remain a number of critical issues in screening libraries consisting of large mixtures of compounds.

By contrast, many other practitioners are using a method illustrated in FIG. 2 called parallel, or robotic, synthesis. This practice simply involves performing a series of individual reactions in separate vessels. Using traditional manual organic synthesis a chemist can synthesize only about 50 compounds per year. By the use of robots, which can perform multiple reactions simultaneously, this procedure can be made more efficient. As shown in FIG. 2, the nine trimers are synthesized in nine reaction vessels in a parallel fashion on the solid support.

One of earliest examples of the parallel method for the synthesis of compounds is the “multipin method” developed by Geysen et al., for combinatorial solid-phase peptide synthesis (Geysen et al.; J. Immunol. Meth. (1987)102:259-274). According to this method, a series of 96 pins are mounted on a block in an arrangement and spacing which correspond to a 96-well microtiter reaction plate, and the surface of each pin is derivatized to contain a terminal linker functional groups. The pin block is then lowered into a series of reaction plates to immerse the pins in the wells of the plates where coupling occurs at the terminal linker functional groups, and a plurality of further reactions are carried out in a similar fashion. Reagents varying in their substituent groups occupy the wells of each plate in a predetermined array, to form a unique product on each pin. By using different combinations of substituents, one achieves a large number of different compounds with an array of central core structures.

Another type of solid phase parallel synthesis method is the diversomer approach from Park-Davis group (DeWitt, S. H.; et al. Proc. Natl. Acad. Sci. USA, 90, 6909, 1993). It was designed for the synthesis of small organic molecules. The solid support was placed into porous tubes immersed into tubes containing the various reagents which pass through the porous walls to contact the solid phase support.

A related method of synthesis uses porous polyethylene bags (Tea Bag method) containing the functionalized solid phase resins (Houghton, R. A., et al., Nature, 354, 84-86, 1991). These bags of resin can be moved from one reaction vessel to another in order to undergo a series of reaction steps for the synthesis of libraries of products.

As a consequence of the development of the efficient automation equipment and processes, the parallel synthesis technique has now become the most extensively used method in combinatorial chemistry. However, the libraries created using the parallel method (one compound per vessel) usually require more steps than those created using other combinatorial syntheses. As a result, more time is required to synthesize a comparable size library than would be required using other combinatorial techniques, such as the portioning-mixing method discussed above.

In view of the above, the field of pharmaceutical and agricultural research has a strong need for highly flexible technologies to generate a large number of novel classes of compounds for screening and clinical testing.

An object of this invention is to provide an exceptionally flexible technology for high throughput parallel synthesis of combinatorial libraries.

Another object is to provide a method for efficiently forming combinatorial libraries in which the compounds are formed substantially in mol ratios of 1 to 1.

Yet another object of the invention is to provide a method for forming a variety of different compounds and for recovering the various compounds in a pure state without contamination by the other formed compounds.

### SUMMARY OF THE INVENTION

The present invention is directed to a general method for high-speed parallel synthesis of combinatorial libraries. In accordance with the invention multiple different resins are combined in the same reaction vessel in which a plurality of chemical reactions are carried out to create multiple compounds which are then sequentially cleaved from the resins under the appropriate cleavage conditions. As used herein resins are considered different when they exhibit different chemical activity in the presence of cleaving or releasing agents. Thus, resins having different polymeric backbones but the same linking molecule will exhibit different chemical activity. Likewise, resins having the same or substantially the same polymeric backbone but a different linker will also exhibit a different chemical activity in the presence of cleaving or release agents. Thus, the resins are different when the individual resins have either dissimilar polymeric backbones or dissimilar linkers and thus have a different chemical activity in the presence of a release or cleaving agent from the other resins in the reaction vessel.

### BRIEF DESCRIPTION OF THE DRAWINGS

FIG. 1 illustrates the prior art split synthesis method for forming a combinatorial library;

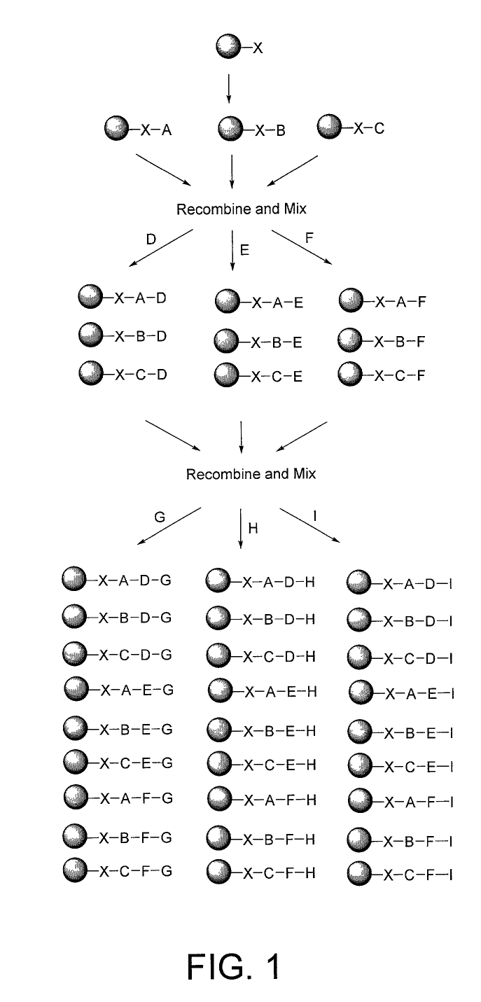

FIG. 2 illustrates the prior art parallel synthesis method for preparing a combinatorial library;

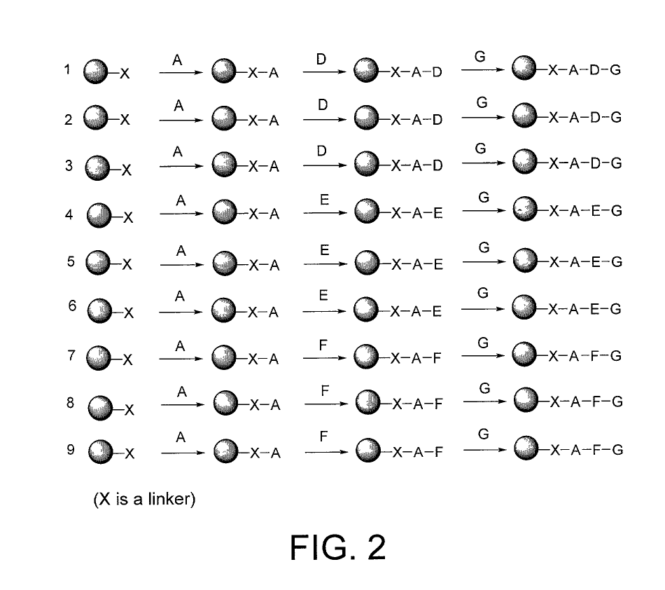

FIG. 3 is a schematic illustration of a conventional parallel synthesis of a combinatorial library utilizing a mono-functional solid support;

FIG. 4 is a schematic illustration of the production of a combinatorial library according to the present invention.

FIG. 5 illustrates in somewhat more detail the method of the invention illustrated by FIG. 4;

FIG. 6 illustrates the addition of functional groups to facilitate selective cleavage through cyclization;

FIG. 7 illustrate the method of the invention where two of three the resins of dissimilar functionality also contain different protecting groups;

FIG. 8 is a reaction scheme described in Example I;

FIG. 9 is a reaction scheme described in Example II;

FIG. 10 is a reaction scheme described in Example III; and

### DESCRIPTION OF THE INVENTION

In accordance with the invention a solid phase method is provided for the high speed production of combinatorial libraries which may contain at least twice the number of compounds as is formed using conventional parallel synthesis techniques. In accordance with the present method two or more resins having dissimilar functionality are combined in the same reaction vessel to provide a solid support on which the library is formed. In this fashion the number of different products that can be formed is the product of the number of dissimilar functionalities forming the solid support. This is to be contrasted with conventional parallel synthesis techniques in which the solid support comprises a single functionality.

As used herein the term “functionality” is defined as the chemical activity exhibited by a resin under a given set of cleavage conditions. Thus, in the case where a solid support formed in accordance with the invention comprises a mixture of at least two dissimilarly functional resins, for example, one from which compounds formed thereon are cleaved under strong acid conditions and the other from which the compounds formed thereon are cleaved under mild conditions, can be used to form twice as many different compounds as a mono-functional solid support conventionally used in parallel synthesis techniques.

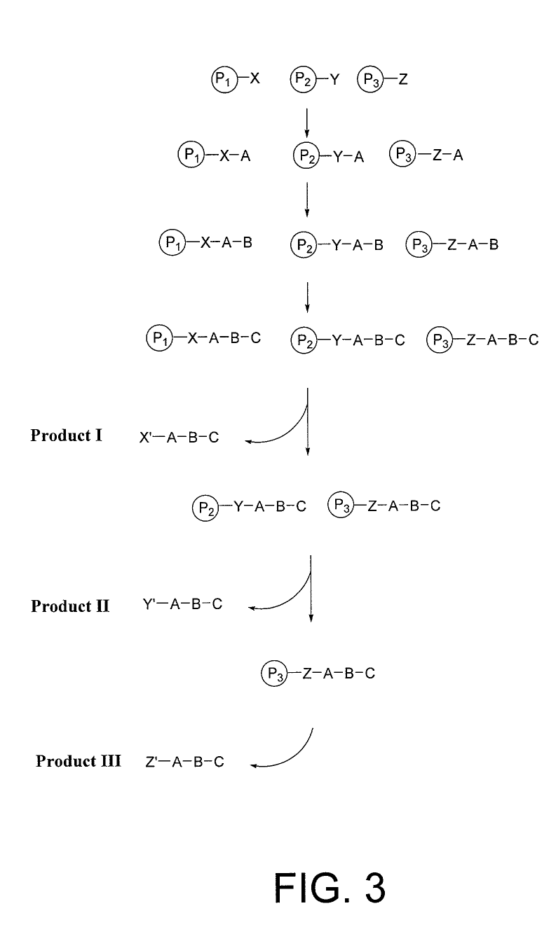

FIG. 3 illustrates the general method of this invention wherein

The solid supports are presented by the following symbol:

\mathring{P₁} \mathring{P₂} \mathring{P₃} . . . Pn

X, Y and Z are independently linkers bearing various functional groups including but not limited to oxygen, sulfur and nitrogen;

A, B, C, D and E are independently organic building blocks, including but not limited to amines, aldehydes, ketones, carboxylic acids, heterocyclic scaffolds, amino acids, carbohydrate moieties, nucleotide;

The covalent forms of A, B, C, D, E and F, such as the intermediates A-B and A-B-C, and the products, such as X′-A-B-C, are independently any organic scaffolds, small organic molecules, include but not limited to heterocycle compounds, and biopolymers such as oligosaccharides, peptides and oligonucleotides.

Referring to FIG. 3 the solid phase support combinatorial synthesis method of the invention is illustrated in which multiple resins (e.g., three resins P₁, P₂ and P₃) containing the linkers X, Y and Z respectively are mixed in the same vessel. It will be understood that, as mentioned above, the linkers X, Y and Z may be the same or different between the resins of different backbones that result in dissimilar functionality. Likewise, the resins may have the same polymeric backbones but different linkers that likewise result in dissimilar functionality between the resins. These are then subjected to the reaction with the first synthon or building block “A”. Subsequent reactions with “B” and “C” synthons generate the trimers. The strategy for selectively and individually releasing the products from the solid support is based on both the activities of the linkers and the nature of the polymers used. The first product is released under conditions that allow only the covalent bond between the polymer support 1 and the linker X to be cleaved while the others are inert. The first product is then recovered from the vessel by suitable well understood washing techniques while the second and third products are immobilized on the solid phase support. By applying the same concept, the second and third products are selectively sequentially cleaved and recovered from the polymer support 2 and 3, respectively in the same vessel. As mentioned above the number of different resins, and thus the number of different compounds formed and recovered, is not limited to three. It is preferred, however, that not more than about six different resins be used in order to allow for the recovery of sufficient quantities of compounds for screening purposes.

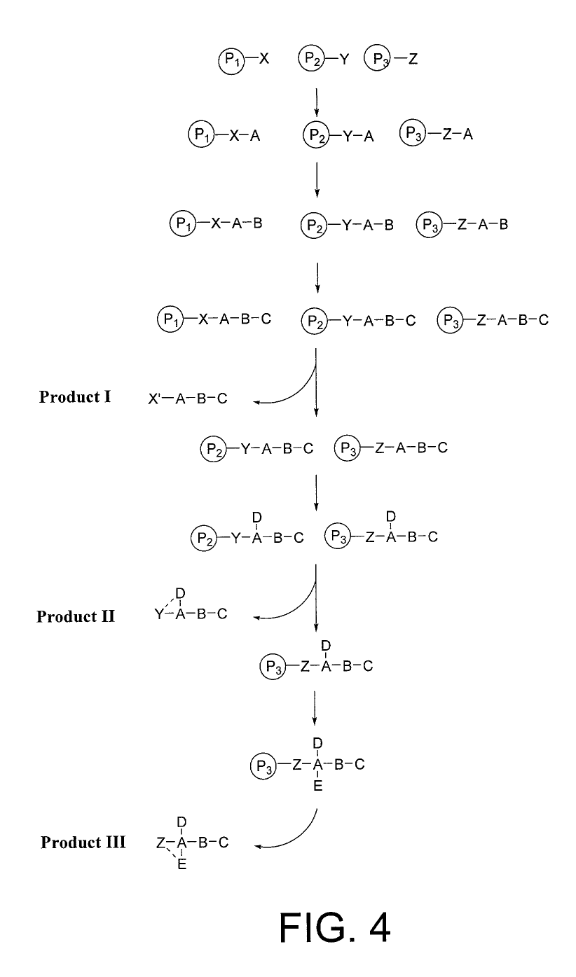

FIG. 4 illustrates the selective releasing of a second product by an additional chemical manipulation that introduces a functional group “D” to facilitate the selective cleavage through cyclization. Using the same concept, the group “E” is introduced to arm the cyclization and releasing the third product.

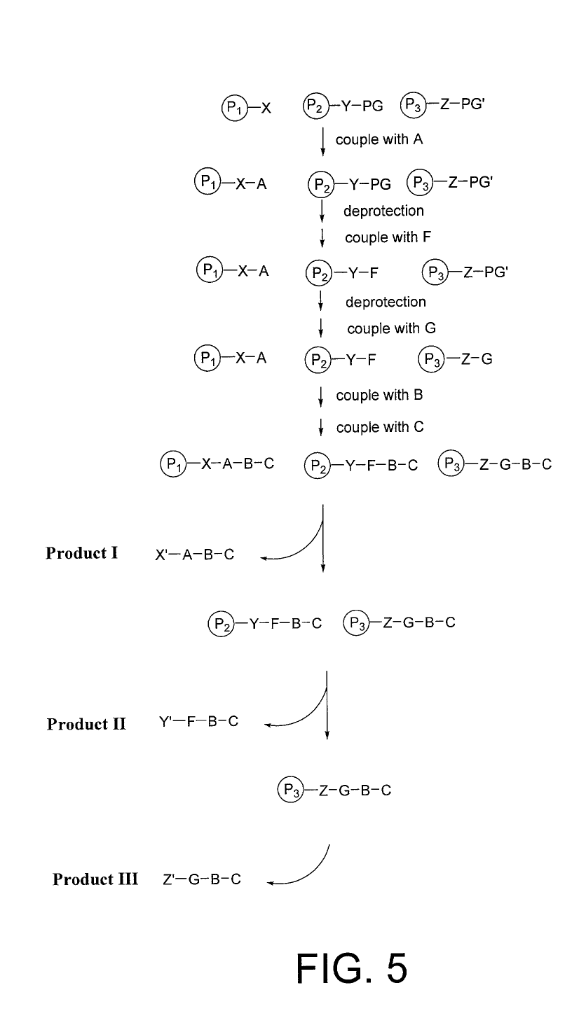

FIG. 5 illustrates a technique in accordance with the invention in which two of the initial multiple resins contain different protecting groups, which are selectively removed after a few chemical reactions are carried out. As shown, the first resin is unprotected and the second and third resins are protected. After the coupling of the building block A to the first resin, the polymer support 2 is selectively deprotected and then reacts with F. The protecting group on the polymer support 3 is removed and reaction with G can be achieved. At this stage, additional building blocks are readily coupled in the same ways as described in the FIGS. 3 and 4. Similarly, the selective cleavage strategy results in the multiple products sequentially released.

In the practice of producing combinatorial libraries the above-discussed approaches can be combined in order to reach the maximum efficiency in creating the molecular diversity. The order of the deprotection, coupling and other chemical reactions with the building blocks can also be altered. The number of the resins used in the same reaction vessel is not limited in this patent.

The ease of purification and automation of solid support synthesis of peptides and non-peptide-based molecules gives several advantages to solid support synthesis over solution chemistry. (Atherton, E.; Sheppard, R C; Solid Phase Peptide Synthesis: A Practical Approach; IRL Press at Oxford University Press: Oxford 1989; Lenzoff, C. C.; Acc. Chem. Res., 1978, 11, 327-333 [non-peptide molecules]). Solid support synthesis of combinatorial libraries has yielded many biologically active compounds (Moos, W. H. et al.; Annu. Rep. Med. Chem., 1993, 28, 315-324; Terrett, N. K.; Gardner, M.; Gordon, D. W.; Kobylecki, R. J.; Steele, J.; Tetrahedron 1995, 51, 8135-73).

Solid support synthesis is carried out on a substrate consisting of a polymer, cross-linked polymer, functionalized polymeric pin, or other insoluble material. These polymers or insoluble materials have been described in literature and are known to those who are skilled in the art of solid phase synthesis (Stewart J M, Young J. D.; Solid Phase Peptide Synthesis, 2nd Ed; Pierce Chemical Company: Rockford. Ill., 1984). Some of them are based on polymeric organic substrates such as polyethylene, polystyrene, polypropylene, polyethylene glycol, polyacrylamide, and cellulose. Additional types of supports include composite structures such as grafted copolymers and polymeric substrates such as polyacrylamide supported within an inorganic matrix such as kieselguhr particles, silica gel, and controlled pore glass.

Such polymers are substituted with linkers that modulate the stability of the linkage to the resin. The linkers incorporate reactive functionalities (A), (e.g. amino, hydroxy, oximino, phenolic, silyl, etc.) for loading of monomers suitable for carrying out a plurality of further reactions to synthesize the desired products (Hemkens, P. H. H.; Ottenheijm, H. C. J.; Rees, D.; Tetrahedron Lett. 1996, 52, 4527-54).

Examples of suitable support resins and linkers are given in various reviews (Barany, G.; Merrifield, R. B. “Solid Phase Peptide Synthesis”, in “The Peptides—Analysis, Synthesis, Biology. Vol 2,” [Gross, E. and Meienhofer, J., Eds.], Academic Press, Inc., New York, 1979, pp 1-284; Backes, B. J.; Ellman, J. A. Curr. Opin. Chem. Biol. 1997. 1, 86.) and in commercial catalogs (Advanced ChemTech, Louisville, Ky.; Novabiochem, San Diego, Calif.). Some examples of particularly useful functionalized resin/linker combinations that are meant to be illustrative and not limiting in scope are shown below:

1. Merrifield resin—Chloromethyl co-poly(styrene-1 or 2%-divinylbenzene) which can be

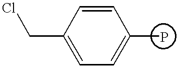

2 Benzhydrylamine copoly(styrene-1 or 2%-divinylbenzene) which referred to as the BHA

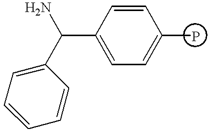

3 Methyl benzhydrylamine copoly(styrene-1 or 2%-divinylbenzene) which is referred to as MBHA and represented as:

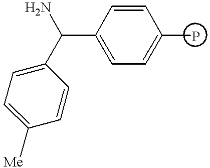

4. Argogel resins

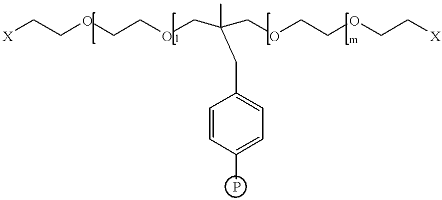

Some additional resins that are useful in specialized situations are:

5. Trityl and functionalized Trityl resins, such as 2-chlorotrityl resin (Barlos, K.; Gatos, D.; Papapholiu, G.; Schafer, W.; Wenqing, Y.; Tetrahedron Lett. 1989, 30, 3947).

6. Sieber amide resin (Sieber, P.; Tetrahedron Lett. 1987, 28, 2107).

7. Wang resin (Wang, S. S.; J. Am. Chem. Soc. 1973, 95, 1328-1333).

8. Oxime resin (DeGrado, W. F.; Kaiser, E. T.; J.Org. Chem. 1982, 47, 3258).

9. Polyoxyethylene grafted (Tentagel) resins (Rapp, W.; Zhang, L.; Habich, R.; Bayer, E. in “Peptides 1988; Proc. 20^{tth} European Peptide Symposium” [Jung, G. and Bayer, E., Eds.], Walter de Gruyter, Berlin, 1989, pp 199-201).

10. Safety catch resins (see resin reviews above; Backes, B. J.; Virgilio, A. A.; Ellman, J. A.; J. Am. Chem. Soc. 1996, 118, 3055-6).

11. Photolabile resins (e.g. Abraham, N. A. et al.; Tetrahedron Lett. 1991, 32, 577).

12. Rink acid resin (Rink, H.; Tetrahedron Lett. 1987, 28, 3787).

13. HPPB-BHA resin (4-hydroxymethyl-3-methoxyphenoxybutyric acid-BHA Florsheimer, A.; Riniker, B. in “Peptides 1990; Proceedings of the 21^{st} European Peptide Symposium” [Giralt, E. and Andreu, D. Eds.], ESCOM, Leiden, 1991, pp 131).

14. Resins with silicon linkage (Chenera, B.; Finkelstein, J. A.; Veber, D. F.; J. Am. Chem. Soc. 1995, 117, 11999-12000; Woolard, F. X.; Paetsch, J.; Ellman, J. A.; J. Org. Chem. 1997, 62, 6102-3).

15. PEGA resins (Bis 2-acrylamidoprop-1-yl polyethyleneglycol crosslinked dimethyl acrylamide and acryloyl sarcosine methyl ester) (Meldal, M.; Tetrahedron Letters 1992, 33, 3077).

Also useful as a solid phase support in the present invention are solubilizable resins that can be rendered insoluble during the synthesis process as solid phase supports. Although this technique is frequently referred to as “Liquid Phase Synthesis”, the critical aspect for our process is the isolation of individual molecules from each other on the resin and the ability to wash away excess reagents following a reaction sequence. This also is achieved by attachment to resins that can be solubilized under certain solvent and reaction conditions and rendered insoluble for isolation of reaction products from reagents. This latter approach, (Vandersteen, A. M.; Han, H.; Janda, K. D.; Molecular Diversity, 1996, 2, 89-96.) uses high molecular weight polyethyleneglycol as a solubilizable polymeric support and such resins are also used in the present invention.

Among the reaction sequences carried out by the method of the present invention is the formation of the amide bond. Many suitable reagents are known to the art to be suitable for this reaction sequence (i.e. Stewart J M, Young J. D.; Solid Phase Peptide Synthesis, 2nd Ed; Pierce Chemical Company: Rockford. Ill., 1984). Among the many useful reagents available, some preferred reagents include dialkylcarbodiimide with an additive such as 1-hydroxy-benzotriazole, particularly diispropylcarbodiimide/1-hydroxy-azabenzotriazole and the like (DIC/HABT); benzotriazol-1-yloxytris-(dimethylamino)-phosphonium hexafluorophosphate (BOP); O-benzotriazolo-N,N,N′,N′-tetramethyluronium hexafluorophosphate (HBTU); Bromo-tris-pyrrolidinophosphonium Hexafluoro-phosphate (PyBrOP), Fmoc amino acid fluorides (Carpino, L. A., et al. 9-Fluoroenylmethyloxycarbonyl (FMOC) Amino Acid Fluorides. Convenient New Peptide Coupling Reagents Applicable to the FMOC/tert-Butyl Strategy For Solution and Solid-Phase Synthesis, J. Am. Chem.Soc., 1990, 112, pp 9651-2) and the like. The degree of steric hindrance, reactivity of the amine, and other well understood factors will be consideed by those skilled in the art to determine which reagent will be most suitable for a particular substrate. However, many of the reagents will give a suitable result for most reactions.

As is conventional, the amide group is protected until it is to be utilized in a reaction sequence. Those skilled in the art will appreciate that any of the wide variety of available amino protecting groups may be used such as tert-Butyloxycarbonyl (Boc), Fluorenylmethyloxycarbonyl (Fmoc), and the like. The choice of a particular protecting group will depend on the specific nature of the substituents and reactions contemplated. Also, more than one type of protecting group may be necessary at any given point in the synthesis (see, e.g., Green, T. and Wuts, P. G. M.; Protective Groups In Organic Synthesis 2^{ND} ED., Wiley, 1991 and references therein).

Cleavage from the solid support can be carried out using one of a number of well-known and convenient procedures (e.g. Stewart, J. M.; Young J. D.; Solid Phase Peptide Synthesis, 2nd Ed; Pierce Chemical Company: Rockford. Ill., 1984; Barany, G.; Merrifield, R. B. “Solid Phase Peptide Synthesis”, in “The Peptides—Analysis, Synthesis, Biology. Vol 2,” [Gross, E. and Meienhofer, J., Eds.], Academic Press, Inc., New York, 1979, pp 1-284). Among these procedures are various acidolytic, based-catalyzed, reductive, photolytic, and self-cleavage techniques.

The conditions used for the popular acidolytic cleavage procedures depends on the particular choice of resin/linker combination used for the synthesis. For example, cleavage may be carried out under conditions utilizing HOAc/CH₂Cl₂ (Rink Acid resin), 5% CF₃CO₂H (2-chlorotrityl resin), 25% CF₃CO₂H (Wang resin), anhydrous HF or mixtures of CF₃SO₃H/CF₃CO₂H (Merrifield resin). Ester resin linkages can be cleaved under nucleophilic conditions to yield, for example, amides (R-NH2/CH₃OH), esters (CH₃OH/Et₃N), hydrazides (N₂H₄/DMF), etc. Catalytic transfer hydrogenation (Pd[OAc] ₂/HCO₂H) has been used to reductively cleave esters from benzylic linkages on resins (Babiker, E.; Anantharamaiah, G. M.; Royer, G. P.; Means; G. E. J. Org. Chem. 1979, 44, 3442-4). A particularly useful nucleophilic cleavage entails a self-cleavage by a functional group in the molecule being synthesized, leading to the formation of a ring system. An example would be attack by an amine function in the compound being synthesized upon the ester linkage to the resin to lead to a new amide function in the target molecule. Such a cleavage step is advantageous in that no harsh reagents are required and, additionally, it may serve as a purification step since impurities lacking the amino function will not be cleaved from the resin. The above examples are merely illustrative of the many suitable cleavage techniques that are documented in review articles such as those above, are well known to those skilled in the art of solid phase synthesis, and are meant to illustrate but not limit the scope of the disclosure.

### EXAMPLES

The following examples are by way of illustration of various aspects of the present invention and are not intended to be limiting thereof.

General Procedures-Reagent Systems and Test Methods

Anhydrous solvents were purchased from Aldrich Chemical Company and used directly. Resins were purchased from Advanced ChemTech, Louisville, Kentucky, and used directly. The loading level ranged from 0.35 to 1.1 mmol/g. Unless otherwise noted, reagents were obtained from commercial suppliers and used without further purification. Preparative thin layer chromatography was preformed on silica gel pre-coated glass plates (Whatman PK5F, 150 Å, 1000 μm) and visualized with UV light, and/or ninhydrin, p-anisaldehyde, ammonium molybdate, or ferric chloride. NMR spectra were obtained on a Varian Mercury 300 MHz spectrometer. Chemical shifts are reported in ppm. Unless otherwise noted, spectra were obtained in CDCl₃ with residual CHCl₃ as an internal standard at 7.26 ppm. IR spectra were obtained on a Midac M1700 and absorbencies are listed in inverse centimeters. HPLC/MS analysis were performed on a Hewlett Packard 1100 with a photodiode array detector coupled to a Micros Platform II electrospray mass spectrometer. An evaporative light scattering detector (Sedex 55) was also incorporated for more accurate evaluation of sample purity. Reverse phase columns were purchased from YMC, Inc. (ODS-A, 3 μm, 120 Å, 4.0×50 mm).

Solvent system A consisted of 97.5% MeOH 2.5% H₂O, and 0.05% TFA. Solvent system B consisted of 97.5% H₂O, 2.5% MeOH, and 0.05% TFA. Samples were typically acquired at a mobile phase flow rate of 2 ml/min involving a 2 minute gradient from solvent B to solvent A with 5 minute run times. Resins were washed with appropriate solvents (100 mg of resin/1 ml of solvent). Technical grade solvents were used for resin washing.

### Example I

Simultaneous Synthesis of Peptoid Carboxylic Acids and Peptoid Amides in the Same Reaction Vessel using Combined Resin Method

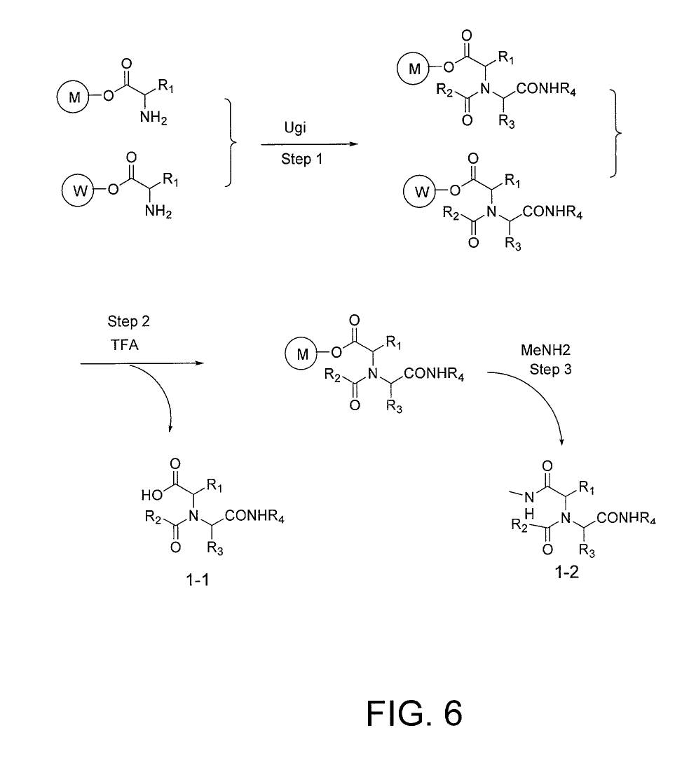

The following example taken with FIG. 6 illustrates a procedure for the preparation of 2-[Benzoyl-(1-tert-butylcarbamoyl-3-phenyl-propyl)-amino]-3-phenyl-propionic acid (Compound 1-1) and N-(1-tert-butylcarbamoyl-3-phenyl-propyl)-N-(butyl-carbamoyl-2-phenyl-ethyl)-benzamide (Compound 1-2) by the Ugi reaction utilizing a combination of dissimilar resins in a single vessel. FIG. 6 illustrates the procedure described herein. As used in the example and in FIG. 6, R₁=benzyl, R₂=benzyl, R₃=phenyl, R₄=phenethyl and R₅=n-butyl.

A mixture of Phe-Wang resin (0.16 mmol) and Phe-Merrifield resin (0.11 mmol) was taken up in 4 mL of 3:1 THF:MeOH. To this was added 330 mg (2.7 mmol) of benzoic acid, 2.7 mmol of hydrocinnamaldehyde and 2.7 mL of 1M solution of n-Butyl isocyanide in methanol. The reaction mixture was shaken for 2 days and then filtered and washed with THF (2×), DCM (2×), MeOH (2×) and dried in vacuo. The resin mixture was subjected to the first cleavage with 2 mL of 25% TFA in DCM for 45 min. The resin was filtered, and the filtrate was concentrated in vacuo following addition of 1:1 acetonitrile:water. The resin was washed with 10% DIPEA in DCM (2×), DMF (2×), DCM (2×), MeOH (2×) and dried, then subjected to the second cleavage with 2 mL of 1:1 MeNH₂:THF and shaken overnight The resin was filtered, and the filtrate was concentrated in vacuo. The concentrate from TFA cleavage was analyzed by LC-MS [retention time: 3.52 min; MS (ES) m/e: 487 (M+H⁺)]. Purification on a preparative silica gel TLC plate using 4:4:1 Hexane:EtOAc:MeOH gave 35 mg of the desired product in 48% yield. The concentrate from MeNH₂ cleavage was analyzed by LC-MS MS [retention time: 3.38 min; MS (ES) m/e: 496 (M+H⁺)]. Purification on a preparative silica gel TLC plate using 4:4:0.5 Hexane:EtOAc:MeOH gave 32 mg of the desired product in 62% yield.

### Example II

Simultaneous Synthesis of Two Heterocyclic Compounds in the Same Reaction Vessel Using Combined Resin Method

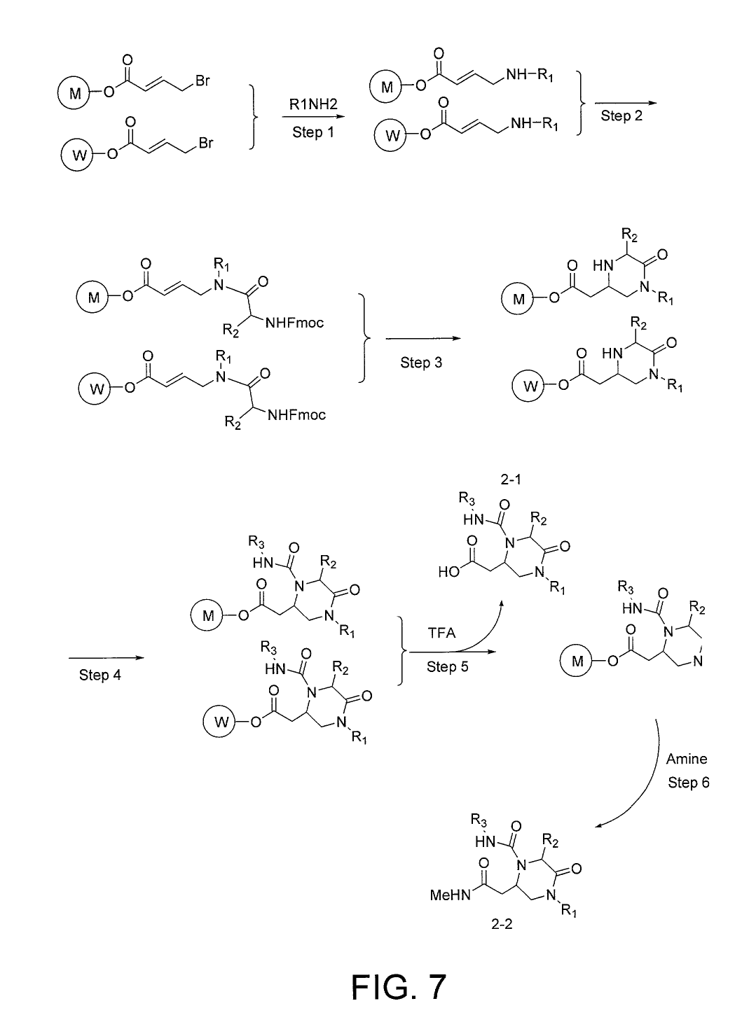

The following example taken with FIG. 7 illustrates a procedure for the preparation of [4,6-(S)-Dibenzyl-1-cyclohexylcarbamoyl-5-oxo-piperazin-2yl]acetic acid (Compound 2-1,) and [2-(S)-,4-Dibenzyl-6-methylcarbamoylmethyl-3-oxo-piperazin-1-yl]-carboxylic acid cyclohexylamide (Compound 2-2) utilizing a combination of dissimilar resins in a single vessel. FIG. 7 illustrates the procedure described herein.

Step 1: Displacement of Bromide

A mixture of Wang resin and Merrifield resin in a ratio 1/1 (300 mg) was suspended in a solution of benzylamine [0.5M] in NMP (8 mL) and shaken for 45 min at room temperature. After filtration, the resulting mixture was washed with 2×10 mL of DMF, 3×10 mL of DCM/MeOH, 2×10 mL of DCM then dried under nitrogen. IR 1718 cm⁻¹.

Step 2 Acylation

To the resin were added Fmoc-phenylalanine (10 eq), DIC (10 eq), and DMF (3 mL/100 mg of resin). The resulting mixture was shaken for 24 h at room temperature. After filtration, the resin was washed by 2× DMF (3 mL/100 mg of resin), 2× DCM/MeOH, 2× DCM then dried under nitrogen.

Step 3: Deprotection and Cyclization

The resin was suspended in a solution of piperidine (20%) in DMF (3 mL/100 mg of resin) and shaken for 2×30 min. After filtration, the resin was washed by 2× DMF (3 mL/100 mg of resin), 2× DCM/MeOH, 2× DCM then dried under nitrogen. IR 1734 cm⁻¹.

Step 4: Formation of Ureas

The resin was suspended in a solution of cyclohexyl isocyanate [0.5M] in DCE (3 mL/100 mg of resin) and shaken for 24 h at room temperature. The resin was filtered and washed by 2× DMF, 2× DCM/MeOH, 2× DCM then dried under nitrogen.

Step 5: Cleavage of the first product

The resin was suspended in a mixture of TFA (25%) in DCM (3 mL/100 mg of resin) and shaken for 30 min. After filtration, the resin was washed by 2× DCM (3 mL/100 mg of resin). The volatile materials were removed under reduced pressure. The remaining material was subjected to purification by treatment with TMSCH₂N₂ resulting in the recovery of 25 mg of pure desired compound (39%, based on 0.9 mmol/g loading) as a mixture of two isomers with a 2:1 ratio.

Analysis of the product produced the following data:

MS (ES) m/e (relative intensity): 478 (M+H⁺, 100), 353 (40)

1H NMR (a mixture of two isomers of the corresponding

methyl esters, CDCl₃) δ7.40-7.02 (m,10H), 4.90 (d, 1H), 4.76 (d, 1H), 4.71 (dd, 1H), pb 4.55 (dd, 1H), 4.50 (m, 1H),

4.32 (d, 1H), 4.31 (d, 1H), 4.08 (d, 1H), 4.02 (m, 1H), 3.73 (m, 2H), 3.62 (m, 1H), 3.57 (s, 3H), 3.54 (dd, 1H), 3.48 (s, 3H), 3.43 (dd, 1H), 3.40 (dd, 1H), 3.19 (dd, 1H), 3.09 (dd, 1H), 3.04 (dd, 1H), 2.87 (dd, 1H), 2.48 (dd, 1H), 2.20 (m, 1H), 2.17 (dd, 1H), 1.97 (dd, 1H), 1.95-1.80 (m, 2H), 1.71-1.57 (m, 3H), 1.41-1.25 (m, 2H), 1.10 (m, 1H).

Step 6: Cleavage of the Second Product

The resin was suspended in a 1:1 mixture of methylamine (40% in H₂O)/THF (3 mL per 100 mg of resin) and shaken for 24 h. After filtration, the resin was washed by 2× DCM (3 mL/100 mg of resin). The volatile components were removed under reduced pressure to afford 69 mg of remaining product. 35 mg of pure desired compound was isolated as a mixture of two isomers with a 2:1 ratio (55%, based on 0.9 mmol/g loading) using TLC plate purification. Analysis of the recovered product resulted in the following:

MS (ES) m/e (relative intensity): 477 (M+H⁺, 70), 352 (100).

1H NMR (a mixture of two isomers, CDCl₃) δ 7.40-7.05 (m, 10H), 5.24 (d, 1H), 5.15 (d, 1H), 4.98 (d, 1H), 4.90 (d, 1H), 4.79 (dd, 1H), 4.66 (dd, 1H), 4.58 (br, 1H), 4.35 (m, 1H), 4.22 (d, 1H), 3.77 (dd, 1H), 3.70 (d, 1H), 3.61 (m, 1H), 3.53 (dd, 1H), 3.43 (m, 1H), 3.37 (dd, 1H), 3.10 (dd, 1H), 3.02 (dd, 1H), 2.59 (d, 3H), 2.50 (d, 3H), 2.26 (d, 1H), 2.02 (d, 1H), 1.94 (d, 1H), 1.82-1.55 (m, 4H), 1.37-1.25 (m, 2H), 1.13 (m, 1H).

### Example III

Simultaneous Synthesis of Benzodiazepinone Carboxylic Acids and the Corresponding N-methylamides in the Same Reaction Vessel

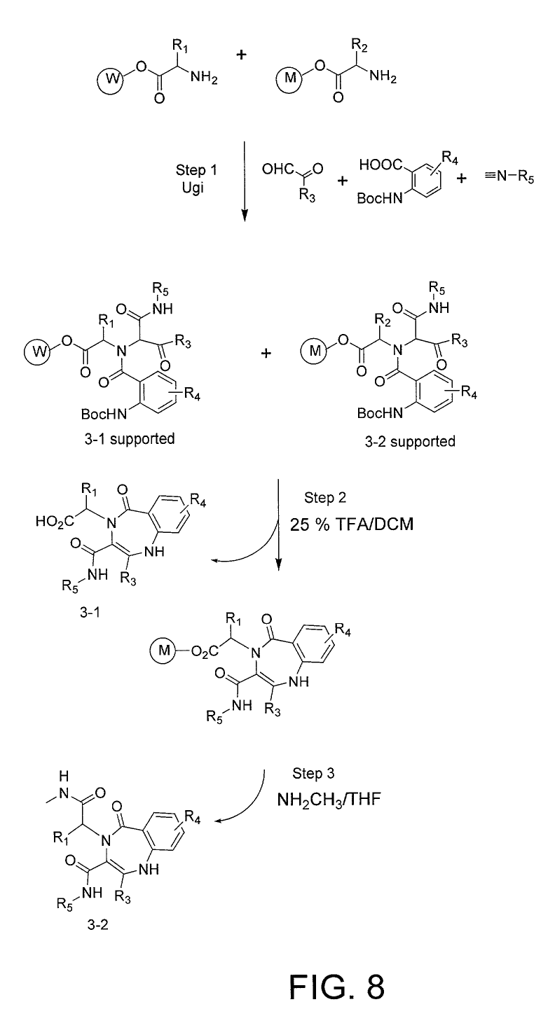

The following example taken with FIG. 8 illustrates a procedure for the preparation of 3-[3-tert-butylcarbamoyl-2-(4-fluoro-phenyl)-5-oxo-1,5-dihydrp-benzo[e][1,4]-diazapin-4-yl]-propionic acid (Compound 3-1,) and [2-(4-Fluoro-phenyl)-4-(2-methylcarbamoyl-ethyl)-5-oxo-4,5-dihydro-1H benzo[e]-[1,4] diazepin-3-yl]-carboxylic acid tert-butylamide (Compound 3-2) utilizing a combination of dissimilar resins in a single vessel. FIG. 8 illustrates the procedure described herein. As used in the example and in FIG. 8, R₁=benzyl, R₂=benzyl, R₃=p-fluorophenyl, R₄=H and R₅=t-butyl.

Step 1: Preparation of Ugi Products 3-1 and 3-2 on Solid Support

A mixture of deprotected phenylalanine Wang resin (200 mg, 0.16 mmol) and phenylalanine Merrifield resin (300 mg, 0.16 mmol ) was placed in a 40 mL glass reaction vial. A 1.0 M solution of p-fluorophenylglyoxal in THF (3.2 mL) was added, followed by addition of 1.6 mL of 1.0 M solution of ZnCl₂ in diethyl ether, 3.2 ml of 1.0 M solution of N-Boc-2-aminobenzoic acid in 1:1 MeOH/THF, and 3.2 mL of 1.0 M solution of cyclohexyl isocyanide in methanol. The resulting mixture was then shaken at room temperature for two days. The resin was filtered, and washed with DMF (2×), MeOH (3×) and DCM (3×). The resin was dried in vacuum at room temperature.

Step 2: Deprotection Cyclization and Selective Cleavage of Product 3-1

The above obtained resins (200 mg) were treated 25% TFA in DCM (2 mL) at room temperature for 30 min. The resins were filtered and washed twice with 1.0 ml of 5% TFA in DCM. The combined TFA filtrates was concentrated down to give product 3-1.

Step 3: Cleavage of Product 3-2

After the TFA treatment, the resins were washed with 2.0 ml of DCM followed with MeOH. Traces of TFA were removed by washing with 2.0 ml of 1.0 M of DIEA in DCE for 10 minutes then it was washed again three times with 2.0 ml of DCM followed by MeOH.

Compound 3-2 was cleaved from the Merrifield resin by the treatment with 2.0 ml of 1:1 40% NH₂CH₃ in water/THF at room temperature overnight. The cleavage solution was filtered and the resins were washed with 2.0 ml of THF followed with MeOH. The combined solutions were evaporated in vacuum to give compound 3-2 which analyzed as follows:.

¹H NMR (CDCl₃, CD₃OD): δ 10.2 (br, 1H), 7.82 (t, 1H), 7.56 (dd, 1H), 7.46 (t, 1H), 7.42 (dd, 1H), 7.29 (m, 1H), 7.18 (m, 1H), 6.97-6.71 (m, 6H), 6.64 (m, 1H), 5.46 (br, 1H), 5.0 (br, 1H), 3.72 (m, 1H), 3.37 (m, 1H), 3.11 (m, 1H), 2.75 (m, 1H), 1.59 (m, 2H), 1.43 (m, 2H), 1.1-0.9 (m, 6H).

Control Experiment

Under the same reaction conditions as described above, compounds 3-1 and 3-2 were synthesized in separate reaction vessels using Phe-Wang resin and Phe-Merrifield resin respectively. High yields of Products 3-1 and 3-2 were recovered from the respective reaction vessels. Analysis of the products showed that the composition and purity of the products produced by the method of this invention compared to the same products produced by the conventional solid support method was the same.

### Example IV

Simultaneous Synthesis of Three Compounds in the Same Reaction Vessel

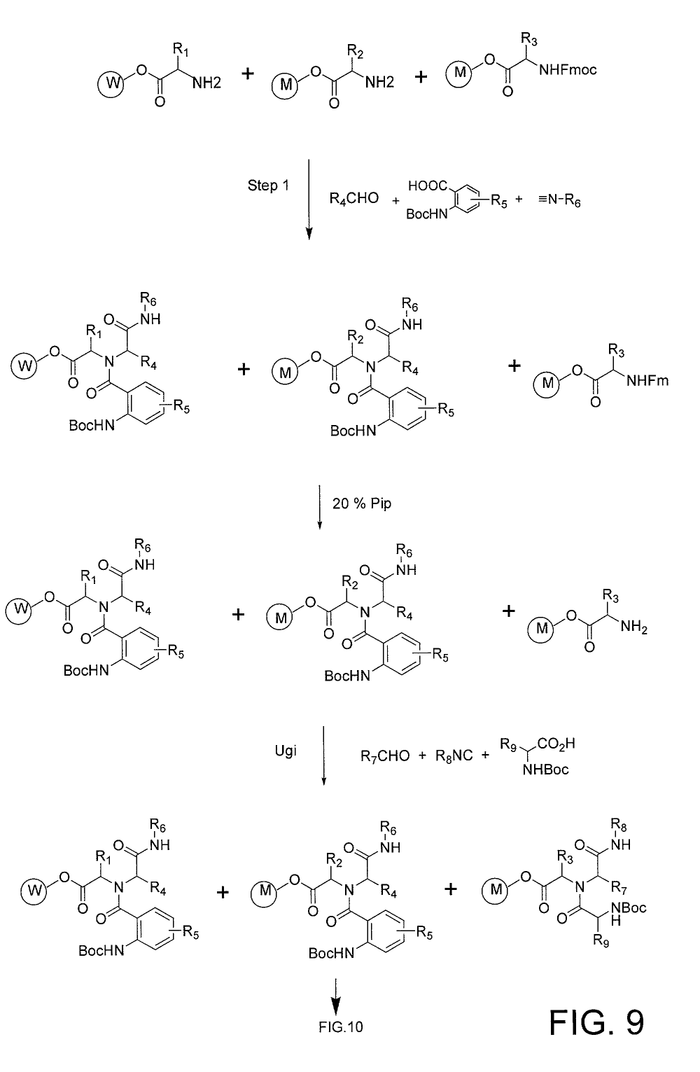

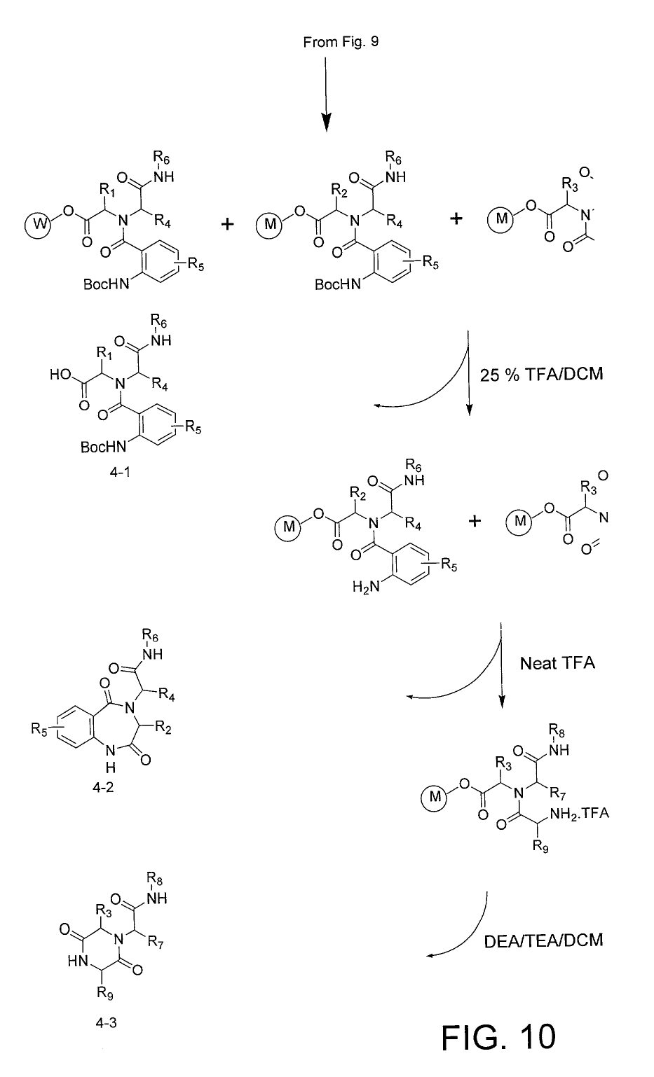

The following example taken with FIGS. 9 and 10 illustrates the simultaneous synthesis of three small molecules, 2-[(2-amino-benzoyl)-(1-tert-butyl carbamoyl-3-phenyl-propyl)-amino]-propionic acid (compound 4-1), a benzodiazepindione, 2-(3-Benzyl-2,5-dioxo-1,2,3,5-tetrahydro-benzo[e][1,4]diazepin-4-yl)-N-tert-butyl-4-phenyl butyramide (Compound 4-2) and a piperazine-2,5-dione, 2-(2-Benzyl-5-isobutyl-3,6-dioxo-piperazin-1-yl)-N-tert-butyl-4-phenyl-butyramide (Compound 4-3). Starting from a mixture of three different aminoacyl resins including Ala-Wang resin, Phe-Merrifield resin and Fmoc-Phe-Merrifield resin, the Ugi reactions on the two free amine containing aminoacyl resins (Ala-Wang and Phe-Merrifield resin) were carried out. Subsequently, the Fmoc-Phe-Merrifield resin was deprotected and the second Ugi reaction was followed. The selective cleavage of the three products was achieved in high yields and purity. As shown in FIGS. 9 and 10, R₁=Me, R₂=benzyl, R₃=benzyl, R₄=phenethyl, R₅=H, R₆=t-butyl, R₇=phenethyl, R₈=t-butyl and R₉=iso-butyl

Step 1: The First Ugi Reaction

An equimolar aminoacyl resin mixture was prepared by mixing 200 μmol of Ala-Wang resin, Phe-Merrifield resin and Fmoc-Phe-Merrifield resin. The combined resin mixture was placed in a 40-ml glass vial. A 1.0 M solution of 3-phenylpropionaldehyde in THF was added to the resins followed by addition of 4.0 ml of 1.0 M solution of 2-N-Boc-aminobenzoic acid in 1:1 MeOH/THF and 4.0 ml 1.0 M solution of t-butyl isocyanide in MeOH. The mixture was shaken at room temperature for 2 days. The resins were filtered, and washed twice with 5 ml of DMF, 5 ml of DCM followed with 5 ml of MeOH (3×). The resins were dried in vacuum at room temperature.

Step 2 and Step 3: Deprotection of the Fmoc-Phe-Merrifield Resin and the Second Ugi Reaction

The Fmoc-Phe-Merrifield resin in the resin mixture was deprotected by treatment with 20% piperidine in DMF at room temperature for 30 min. The resin mixture was washed as above and then the 2^{nd} Ugi reaction was performed using the same aldehyde and isocyanide as above, the carboxylic acid (Boc-Leu-OH) was used to form the corresponding resin bound Ugi product.

2.0 ml of 1.0 M 3-phenylpropionaldehyde solution in THF was added to the resin mixture followed by 2.0 ml of 1.0 M solution of Boc-Leu-OH in 1:1 MeOH/THF and 2.0 ml 1.0 M solution oft-butyl isocyanide in MeOH. The reaction mixture was diluted with 2.0 ml of 1:1 solution of MeOH/THF and then it mixed on a shaker station for 48 hrs at room temperature.

The liquid phase was separated by filtration. The resins containing the three Ugi products were washed in the way as described above and dried in vacuum.

Step 4: Cleavage of Compound 4-1

The above obtained resin mixture (200 mg ) was treated with 2.0 ml of 25% TFA in DCM. Under this conditions the Boc protection of all three Ugi products was removed and compound 4-1 was cleaved from the resin. The resin was washed twice with 1.0 ml of 5% TFA in DCM. The peptoid carboxylic acid Aᵤ₁ was recovered by the concentration of the cleavage cocktail. The resin mixture was washed twice with 2.0 ml of MeOH followed with the same volume of DCM. Analysis of Compound 4-1 provided the following results:

¹H NMR (mixture of 2 isomers with 2:1 ratio, CDCl₃, CD₃OD): ??7.95 (dd, 1H), 7.86 (dd, 1H), 7.44 (m, 2H), 7.24-7.15 (m, 4H), 7.10 (m, 2H), 6.96 (dt, 1H), 6.61 (s, 1H), 6.23 (s, 1H), 5.16 (dd, 1H), 5.03 (dd, 1H), 4.61 (q, 1H), 4.41 (q, 1H), 2.52 (m, 2H), 2.2-2.02 (m, 2H), 1.28 (s, 9H), 1.25 (s, 9H), 1.11 (d, 3H).

Step 5: Cleavage of Compound 4-2

The resin was then treated with 2.0 ml of neat TFA for 6 hrs at room temperature. Under this condition the Ugi product Bᵤ₁ was selectively cyclized to release the benzodiazpine -2,5-dione B from the resin. The resin was filtered and washed twice with 2.0 ml of 5% TFA. Compound 4-2 was then obtained by evaporation in vacuum. Analysis of Compound 4-2 gave the following results:

¹H NMR (mixture of 2 isomers with 2:1 ratio, CDCl₃, CD₃OD): δ 8.24 (br, 1H), 8.01 (dd, 1H), 7.93 (dd, 1H), 7.51 (m, 1H), 7.23-7.08 (m, 10H), 6.98 (m, 2H), 6.68 (br S, 1H), 6.2 (br s, 1H), 5.09 (dd, 1H), 4.98 (dd, 1H), 4.76 (dd, 1H), 4.62 (dd, 1H), 4.34 (dd, 1H), 3.94 (dd, 1H), 3.70 (dd, 1H), 2.72 (dd, 1H), 2.64-2.47 (m, 2H), 2.22-2.0 (m, 2H), 1.37 (s, 9H), 1.32 (s, 9H).

Step 6: Cleavage of Compound 4-3

Finally, the resin was treated with 2.0 ml of a 1:1:1 mixture of DEA:TEA:DCM for 6 hrs at room temperature. Under these conditions a third product was cleaved from the resin. The standard filtration and washing followed by concentration of the filtrate resulted in the recovery of a product that upon analysis proved to be Compound 4-3. The results of the analysis are set forth below.

¹H NMR (CDCl₃): δ 8.21 (br, 1H), 7.25-7.10 (m, 10H), 4.62 (dd, 1H), 4.33 (dd, 1H), 3.70 (dd, 1H), 3.21 (dd, 1H), 3.10 (dd, 1H), 2.67-2.34 (m, 5H), 1.87 (m, 1H), 1.37 (s, 9H), 1.01 (ddd, 1H), 0.73 (d, 3H), 0.69 (d, 3H).

Control Experiments

Compounds 4-1, 4-2 and 4-3 were resynthesized individually, in a set of three controlled experiments starting from the corresponding resin. They were synthesized from Ala-Wang resin, Phe-Merrifield resin and Fmoc-Phe-Merrifield resin, respectively. The conditions employed in the syntheses were identical as of the synthesis on the combined resin mixture. Each compound was cleaved by its specific cleavage method as described above.

Analysis of the three compounds obtained from the controlled experiments and the combined resin method showed that the compounds formed by the combined resin method of the invention gave yields and purity essentially the same as the compounds formed by the conventional solid support resin synthesis of the controls. From the foregoing description and examples it has been shown combinatorial libraries of different compounds are prepared utilizing different resins combined in a single container of the solid support. Combining the different resins in the manner taught herein provides for improved efficiency in the synthesis of compounds by solid support techniques, particularly when employing robotic methods to the synthesis. The present method allows for selective sequential cleavage and recovery of the different compounds without contamination of a desired product by the other products formed during the synthesis.

Having defined the invention, we claim:

1. A method for the preparation of a combinatorial library of at least two dissimilar products comprising the steps of:
a. forming a solid support substrate consisting of at least two resins having dissimilar functionality, said resins containing a linker;
b. contacting said solid support substrate with a synthon selected from the group consisting of monomers, oligomers and oligonucleotides under conditions to couple said synthon and said linker;
c. contacting said solid support substrate containing said synthon with a first cleaving agent under cleavage conditions to cleave the bond between only one of said resins and its linker to release a first product comprising said coupled synthon;
d. recovering said first product from said reaction vessel;
e. contacting said solid support with a second cleaving agent under second cleavage conditions to cleave the bond between only a second of said resins and its linker to release a second product comprising said coupled synthon; and
f. recovering said second product from said reaction vessel.

2. The method for the preparation of a combinatorial library of dissimilar products of claim 1 wherein step b. is repeated to add additional synthons until products of desired structure and chain length are supported on said solid support.

3. The method for the preparation of a combinatorial library of dissimilar products of claim 1 wherein the number of different resins in said combined resin substrate determines the number of different products produced and steps g and h are repeated in sequence as necessary to recover products from said resins.

4. The method for the preparation of a combinatorial library of dissimilar products of claim 1 wherein said solid support substrate is contacted with a first amine containing synthon to attach an amine group, said amine group is protected and said amine group is deprotected for reaction with a second synthon.

5. The method for the preparation of a combinatorial library of dissimilar products of claim 1 wherein said solid support substrate comprises at least two resins having different polymeric backbones.

6. The method for the preparation of a combinatorial library of dissimilar products of claim 1 wherein said combined resin substrate comprises at least two resins having different reactive functionaries substituted thereon as linkers,

7. The method for the preparation of a combinatorial library of dissimilar products of claim 7 wherein said linkers are selected from the group of reactive functionaries consisting of amino, hydroxy, oximino, phenolic, silyl, carboxylic oxo, and the like.

8. The method for the preparation of a combinatorial library of dissimilar products of claim 1 wherein said combined resin substrate comprises a mixture of Phe-Wang resin and Phe-Merrifield resins.

9. The method of claim 1 for the preparation of peptoid carboxylic acids and peptoid amides in the same reaction vessel comprising contacting said combined resin substrate with a synthon consisting of benzoic acid, hydrocinnamaldehyde and n-Butyl isocyanide in methanol under conditions to couple said synthon to said resin substrate, contacting said resin substrate with trifluoroacetic acid to cleave a first product from said resin substrate and thereafter recovering said first product, contacting said resin substrate with a mixture of equal parts of methylamide and tetrahydrofuran to cleave a second product from said resin substrate and recovering said second product.

10. The method of claim 9 for the preparation of 2-[Benzoyl-(1-tert-butylcarbamoyl-3-phenyl-propyl)-amino]-3-phenyl-propionic acid having the structure: 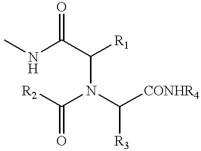
where R₁=benzyl, R₂=benzyl, R₃=phenyl, and R₄=phenethyl.

11. The method of claim 9 for the preparation of N-(1-tert-butylcarbamoyl-3-phenyl-propyl)-N-(butyl-carbamoyl-2-phenyl-ethyl)-benzamide having the structure 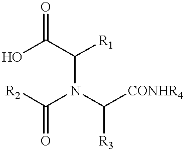
where R₁=benzyl, R₂=benzyl, R₃=phenyl, and R₄=phenethyl.

12. The method of claim 1 for the preparation of a combinatorial library of dissimilar heterocyclic products wherein said combined resin substrate consists of a mixture of Fmoc-phe-ala-Merrifield resin and Fmoc-phe-ala-Wang resin, said method further including the step of deprotecting and cyclizing said resin substrate and thereafter sequentially cleaving and recovering a first heterocyclic product and a second heterocyclic product.

13. The method of claim 13 for the preparation of [4,6-(S)-Dibenzyl-1-cyclohexyl-carbamoyl-5-oxo-piperazin-2yl]acetic acid having the structure 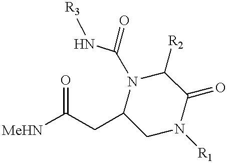
where R₁=benzyl, R₂=benzyl and R₃=phenyl.

14. The method of claim 13 for the preparation of [2-(S)-,4-Dibenzyl-6-methylcarbamoylmethyl-3-oxo-piperazin-1-yl]-carboxylic acid cyclohexylamide having the following structure 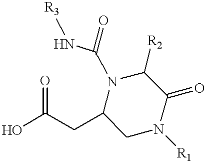
where R₁=benzyl, R₂=benzyl and R₃=phenyl.

15. The method of claim 1 for the simultaneous production of a peptoid carboxylic acid, a benzodiazepindione and a piperazine-2,5-dione comprising forming a solid support resin substrate in a single reaction vessel, said solid support resin substrate consisting of a mixture of Ala-Wang resin, Phe-Merrifield resin and Fmoc-Phe-Merrifield resin, contacting said solid support resin substrate with a synthon consisting of a 1.0M solution of 3-phenylpropionaldehyde in THF, a 1.0 M solution of 2-N-Boc-aminobenzoic acid in 1:1 MeOH/THF and 1.0 M solution of t-butyl isocyanide in MeOH to carry out an Ugi reaction, contacting said solid support resin substrate with piperidine in DMF to deprotect said Fmoc-Phe-Merrifield resin, contacting said solid support resin substrate with a second synthon consisting of 1.0 M solution of t-butyl isocyanide in MeOH, a 1.0 M solution of 2-N-Boc-aminobenzoic acid in 1:1 MeOH/THF and 1.0M solution of 3-phenylpropionaldehyde in THF to carry out a second Ugi reaction, contacting said solid support resin substrate with a mixture of tetrahydrofuran and methylene chloride to deprotect said resins and to cleave as a first product a peptoid carboxylic acid, recovering said first product, contacting said solid support resin substrate with trifluoroacetic acid to cleave as a second product a benzodiazepindione and recovering said second product, finally contacting said solid support resin substrate with a 1:1:1 mixture of diethylamine, triethylamine and methylene chloride to cleave as a third product a piperizine-2,5-dione and recovering said third product.

16. The method of claim 16 for producing 2-[(2-amino-benzoyl)-(1-tert-butyl carbamoyl-3-phenyl-propyl)-amino]-propionic acid having the following structure 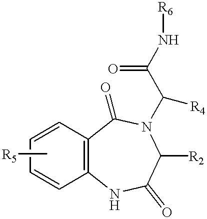
where R₁=Me, R₄=phenethyl, R₅=H, and R₆=t-butyl.

17. The method of claim 16 for producing 2-(3-Benzyl-2,5-dioxo-1,2,3,5-tetrahydro-benzo[e][1,4]diazepin-4-yl)-N-tert-butyl-4-phenyl butyramide having the following structure 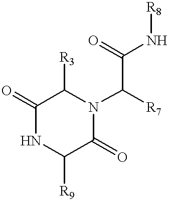
where R₂=benzyl, R₄=phenethyl, R₅=H, R₆=t-butyl.

18. The method of claim 16 for producing 2-(2-Benzyl-5-isobutyl-3,6-dioxo-piperazin-1-yl)-N-tert-butyl-4-phenyl-butyramide having the following structure 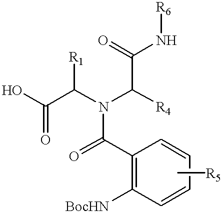
where R₂=benzyl, R₃=benzyl, R₇=phenethyl, R₈=t-butyl and R₉=iso-butyl.
